(19) United States
(12) Patent Application Publication
(10) Pub. No.: US 2008/0044460 A1
(43) Pub. Date: Feb. 21, 2008

(54) Rechargeable Dermal Patch

(75) Inventors: Jean-Claude MILLET, Etoile sur Rhone (FR); Jean-Luc MARTIN, Loriol sur Drome (FR)

Correspondence Address: AKIN GUMP STRAUSS HAUER & FELD L.L.P., ONE COMMERCE SQUARE, 2005 MARKET STREET, SUITE 2200, PHILADELPHIA, PA, 19103, US

(73) Assignee: MILLET INNOVATION, Loriol sur Drome (FR)

(21) Appl. No.: 11/867,164

(22) Filed: Oct. 4, 2007

Related U.S. Application Data
(63) Continuation of application No. PCT/FR2006/000474, filed on Mar. 3, 2006.

(30) Foreign Application Priority Data
Apr. 8, 2005 (FR) 0503499

(51) Int. Cl. A61F 13/02; A61L 15/16

(52) U.S. Cl. 424/448

(57) ABSTRACT

A dermal patch includes a reservoir layer made of a polymer gel provided for receiving a liquid containing an active substance and having a first face to be applied onto the skin of a user in order to diffuse the active substance to the skin. The patch further includes a receiving layer for receiving the liquid containing the active substance and transmitting the liquid to the reservoir layer, and a layer of adhesive holding the receiving layer on a second face of the reservoir layer. The patch has the advantage of being rechargeable as required and can be manufactured without prior incorporation of the liquid containing the active substance into the reservoir layer.

### CROSS-REFERENCE TO RELATED APPLICATIONS

This application is a continuation of International Application No. PCT/FR2006/000474, filed Mar. 3, 2006, which was published in the French language on Oct. 19, 2006, under International Publication No. WO 2006/108933, the disclosure of which is incorporated herein by reference.

### BACKGROUND OF THE INVENTION

The present invention relates to a dermal patch for medical or cosmetic purposes, diffusing more or less slowly an active substance to the skin.

One well-known method involves applying films onto the skin enabling the skin to be given, and as necessary, gradually causing to penetrate into the skin by transdermal process, pharmaceutical (allopathic and/or homeopathic and/or phytotherapy), and/or cosmetic products, particularly aqueous solutions.

One proposal thus made consists in composite films comprising several successive layers including a so-called “reservoir layer,” generally put into contact with the skin and containing the active substance made of one or more treatment products.

In European patent application publication EP 0 412 869 this reservoir layer is made of a silicone polymer matrix, which can contain up to 50% in weight of gelled aqueous phase compared to the total weight of the reservoir layer, the active substance being mixed with the gelled aqueous phase. The polymer is only partially polymerized and is self-adhesive. A frame included in this reservoir layer mechanically strengthens it.

The reservoir layer can be prepared by mixing while shaking the active aqueous phase already gelled into a non-polymerized silicone polymer. It can also be prepared by mixing while shaking a non-polymerized silicone polymer, the active aqueous phase and the gelling agent.

It transpires that this method is limitative in terms of dosage of the active substance, since the latter cannot exceed 0.5 to 1%, and exceptionally up to 5% in weight, of the reservoir layer. In excess of this value, the polymerization enabling a matrix structure to be obtained does not occur. In addition, it has been observed that certain active substances, even in very small proportions, prevent the polymerization.

Moreover, as the active substance is incorporated into the reservoir layer at the time of manufacturing thereof, this method requires the manufacturing of a distinct film for each active substance and for each desired dosage of the active substance, which complicates the manufacturing and management of the stocks of dermal patches. Furthermore, once the active substance in the reservoir layer has been completely absorbed by the user's skin, a patch is not reusable without recycling, which increases its operational cost.

### BRIEF SUMMARY OF THE INVENTION

The present invention aims to remedy these disadvantages by proposing a dermal patch in which the proportion by weight of the active substance is not limited by the polymerization requirements, and into which an active substance can be reintroduced several times, when the quantity of active substance previously introduced into the patch has been absorbed by the user's skin, the reintroduced active substance being either identical to or different from the one previously introduced.

For this purpose, one embodiment of the present invention provides a dermal patch comprising, in addition to a reservoir layer, a receiving layer for receiving a liquid containing an active substance and for transmitting it to the reservoir layer.

More particularly, an embodiment of the present invention provides a dermal patch comprising a reservoir layer made of a polymer gel provided for receiving a liquid containing an active substance and having a first face to be applied onto the skin of a user in order to diffuse the active substance.

According to another embodiment of the present invention, the dermal patch further comprises:

a receiving layer for receiving the liquid containing the active substance and for transmitting the liquid to the reservoir layer, and

a layer of adhesive for holding the receiving layer on a second face of the reservoir layer and which does not hinder the transfer of liquid from the receiving layer to the reservoir layer.

According to one embodiment of the present invention, the reservoir layer comprises a polymethylsiloxane resulting from the polymerization of a mixture of silicone oils.

According to one embodiment of the present invention, the reservoir layer comprises dimethicone or a mixture of dimethicone and vinyl dimethicone.

According to one embodiment of the present invention, the receiving layer is made of a fabric which is elastic at least in one direction.

According to one embodiment of the present invention, the receiving layer is made of a woven-type elastane polyamide fabric.

According to one embodiment of the present invention, the receiving layer has a basis weight on the order of one hundred to about five hundred grams per square meter and a thickness less than one millimeter.

According to one embodiment of the present invention, the reservoir layer has a thickness between 0.2 and 2 mm.

According to one embodiment of the present invention, the first face of the reservoir layer is self-adhesive.

According to one embodiment of the present invention, the layer of adhesive is thin, on the order of one tenth to about five tenths of a millimeter, so as not to prevent the transfer of the liquid comprising the active substance between the receiving layer and the reservoir layer, and to penetrate as little as possible into the receiving layer.

According to one embodiment of the present invention, the layer of adhesive is acetic glue.

The present invention also relates to a method for manufacturing a dermal patch, wherein one embodiment comprises a step of manufacturing a reservoir layer by polymerizing a polymer gel, the reservoir layer being provided for receiving a liquid containing an active substance and diffusing the liquid toward the user's skin.

According to another embodiment of the present invention, the method further comprises the following steps of:

preparing a receiving layer,

assembling the receiving layer and the reservoir layer by means of a layer of adhesive,

the receiving layer being produced with a material capable of receiving the liquid containing the active substance and transmitting it to the reservoir layer, the layer of adhesive being produced so that it does not hinder the transfer of liquid from the receiving layer toward the reservoir layer.

According to one embodiment of the present invention, the method comprises a step of pouring a dose of a liquid containing an active substance onto the receiving layer.

According to one embodiment of the present invention, a liquid containing an active substance is mixed with the silicone gel prior to the polymerization thereof.

According to one embodiment of the present invention, the receiving layer and the reservoir layer are assembled by means of a thin layer of adhesive on the order of one tenth to about five tenths of a millimeter, so as not to prevent the transfer of the liquid comprising the active substance from the receiving layer to the reservoir layer, and to penetrate as little as possible into the receiving layer.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawings:

FIG. 1 is a perspective view of a dermal patch according to one embodiment of the present invention; and

FIG. 2 is a cross-section of the dermal patch according to FIG. 1.

### DETAILED DESCRIPTION OF THE INVENTION

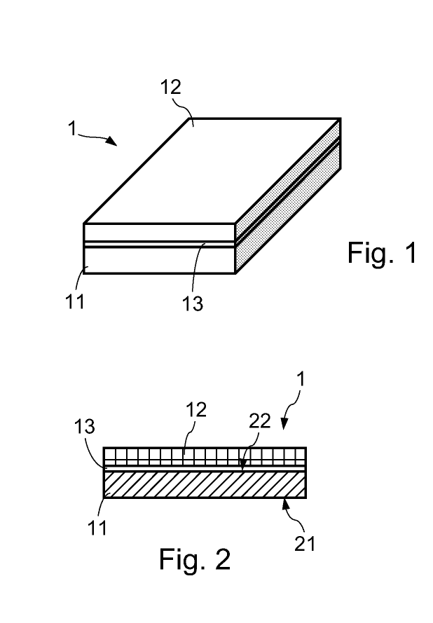

FIGS. 1 and 2 represent a dermal patch 1 comprising a reservoir layer 11 provided for retaining an active substance and diffusing it through the skin of a user, the layer 11 having a face 21 intended to be applied onto the user's skin. According to this embodiment of the present invention, the dermal patch further comprises an external receiving layer 12 provided for receiving a liquid containing the active substance and for transmitting it to the reservoir layer 11 by migration thereof. The reservoir layer 11 and the receiving layer 12 are assembled by means of a layer of adhesive 13, which is produced so as not to prevent the transmission of the liquid containing the active substance from the receiving layer 12 to the reservoir layer 11.

In one example of an embodiment of the present invention, the reservoir layer 11 is produced from a polymerized mixture of two or three silicone oils, which offers the advantage of being self-adhesive on the user's skin. This self-adhesive nature is conventional per se and can be explained by the fact that the polymerization process of the mixed silicone oils is generally not complete, such that after polymerization, the material has a matrix structure containing non-polymerized silicone oils.

The gel obtained has, for example, a matrix structure made of polymethylsiloxane and particularly polydimethylsiloxane, which retains the non-polymerized silicone oils comprising dimethicone or a mixture of dimethicone and vinyl dimethicone. The thickness of the reservoir layer 11 is, for example, between 0.2 and 2 mm.

The receiving layer 12 can be produced in the form of a fabric, which is advantageously elastic at least in one direction, and preferably in two different directions, to enable the patch to be adapted to the shape of the region of the user's skin to which it is applied. This fabric is, for example, woven-type elastane polyamide fabric (comprising weaves of fibers) comprising, for example, 80% polyamide and 20% elastane, and having a basis weight on the order of 180 g/m². Under these conditions, the thickness of the receiving layer is, for example, on the order of 0.6 mm.

The adhesive 13 for assembling the reservoir layer 11 and the receiving layer 12 is preferably an acetic glue, for example a silicone glue with acetic acid. The thickness of the layer of adhesive is preferably low, for example on the order of 0.1 to 0.2 mm, so as not to hinder the migration of the liquid comprising the active substance between the receiving layer and the reservoir layer, and to penetrate as little as possible between the weaves of the fabric forming the receiving layer, so as not to reduce the liquid absorption capacity thereof.

The active substance introduced into the patch comprises a pharmaceutical (allopathic and/or homeopathic and/or phytotherapy) and/or cosmetic composition, such as one or more essential oils having therapeutic and/or cosmetic properties.

The dermal patch according to one embodiment of the present invention is, for example, produced as indicated below.

The reservoir layer 11 is first of all produced from a mixture of silicone oils to obtain a silicone gel, such as PDMS. The structure thus obtained is a matrix structure, which retains the non-polymerized silicone oils. The active substance is not mixed with the silicone oils before polymerization, such that polymerization blocking problems, which can be caused by the liquid containing the active substance if the latter is totally or partially incompatible with a high dosage, are removed. However, if the liquid containing the active substance is compatible with polymerization, it can be mixed with the silicone oils before their polymerization.

A face 22 of the reservoir layer 11, opposite the face 21, is then coated with adhesive 13. Then, the receiving layer 12 is applied to the face 22, so that the layers 11, 12 are assembled after the adhesive 13 has dried. As indicated above, the adhesive 13 is used in such a quantity that it does not pass through the weaves of the fabric after the application of the two layers against each other.

In one alternative embodiment, the adhesive is deposited on the receiving layer 12 and not on the reservoir layer 11. The adhesive can also be deposited both on the receiving layer 12 and the reservoir layer 11, subject to the final thickness of adhesive obtained and provided that the adhesive does not then hinder the migration of the liquid.

Finally, the face 21 of the reservoir layer 11 is preferably protected by an anti-adhesive film, such as a polyethylene film, intended to be removed before applying the patch onto the skin.

The shape and dimensions of the dermal patches according to embodiments of the present invention are adapted to the shape and area of the zone of the user's body to which the patch must be applied. Generally, the patch has a square or rectangular shape, with a length and width of about three centimeters to twelve centimeters. The patch can also have the shape of a set square, for example when it is intended to be applied to the shoulder. In addition, it can be cut into the desired dimensions at the time it is applied onto the user's body.

The dermal patch according to one embodiment of the present invention can also be obtained by manufacturing a large composite film by assembling a receiving layer and a reservoir layer, then by cutting the film to obtain patches of the desired dimensions. The receiving layer can thus be distributed in a roll above the reservoir layer, one and/or the other of these layers previously being coated with adhesive.

The liquid containing the active substance chosen is applied to the visible face of the receiving layer 12 by means enabling the quantity of liquid provided to be dosed, for example a pipette or single-dose packaging. The quantity of liquid transferred into the patch depends on two parameters, i.e. the quantity of liquid the receiving layer 12 is capable of absorbing at a time, and the quantity of active substance it is desirable to transfer to the user's skin.

It can be observed that the liquid applied to the patch spreads virtually instantly throughout the entire receiving layer 12, which is thus used as an intermediate reservoir in which the liquid is first of all retained. In the example of an embodiment described above, the liquid is thus absorbed by the frame of the fabric, which has not been filled in by the adhesive, under the effect of the relative surface tension between the liquid and the thread of the fabric. The liquid is thus transferred from the receiving layer 12 to the reservoir layer 11 over their entire interface. Therefore, it can be seen that the liquid migrates very rapidly toward the reservoir layer 11, then through the reservoir layer toward the self-adhesive face 21 intended to be put into contact with the user's skin. Therefore, the receiving layer 12 tends to dry rapidly.

In the reservoir layer 11 the liquid containing the active substance is mixed with the non-polymerized silicone oils, which are spread in the entire reservoir layer and retained by the polymerized matrix.

The quantity of active substance likely to be provided is important, since the latter should not risk preventing the formation of the polymer gel constituting the reservoir layer 11 by partial polymerization. However, the liquid containing the active substance is preferably miscible with silicone oils. A hydrophobic and non-polar liquid should preferably be used. If it is not possible to naturally dissolve the active substance in a hydrophobic liquid, micro- or nano-emulsions can be produced from the active substance, or support molecules, such as cyclodextrins, can be used to lock the active substance in a hydrophobic mixture.

The greater the quantity of active substance introduced into the reservoir layer, the faster the active substance is transferred to the user's skin. If certain essential oils are used, the diffusion into the skin or any compatible receiving surface starts a few dozen seconds after applying the patch. For certain pathologies, an effect on the user after approximately only ten minutes of applying the patch can be observed. It can also be observed that the patch continues to diffuse the active substance after about ten days of continuous application.

It will be understood that the nature of the liquid bearing the active substance can also influence the performance of the dermal patch, in terms of speed of absorption of the liquid by the receiving layer 12 and speed of migration of the latter into the reservoir layer 11. The liquid used must particularly be compatible with the material forming the reservoir layer, as those skilled in the art well know, to be properly absorbed by the latter.

A dermal patch according to embodiments of the present invention may simultaneously or successively receive several different liquids containing complementary active substances in predefined proportions.

Therefore, for example, one or more anti-inflammatory drugs can be simultaneously or successively applied together with a composition intended to facilitate the penetration of the liquids into the skin. The patch can thus be recharged several times for a long treatment, either with the same liquid or with liquids containing different active substances for complementary treatments.

As the speed of diffusion to the skin of an active substance inserted into the dermal patch according to embodiments of the present invention depends particularly on the concentration of the active substance in the reservoir layer, two active substances can be introduced into the reservoir layer with different dosages, so as to combine the effect of an active substance introduced in a low quantity, which is thus diffused slowly, with the principle active introduced in more significant quantity, which is more rapidly diffused.

It will be understood by those skilled in the art that various alternative embodiments of the dermal patch according to the present invention are possible, particularly as far as each of its constituent materials is concerned.

Therefore, the present invention does not exclude non self-adhesive or insufficiently adhesive compositions from being used to hold the patch on the skin. In such a case, the patch can be arranged on a self-adhesive material of a sticking plaster type, having on its rear face a window enabling the liquid to be introduced onto the receiving layer 12.

Furthermore, the reservoir layer 11 may comprise several layers of polymer gel assembled without any adhesive, by simple mutual contact.

Moreover, the receiving layer 12 may be made of a non-woven fabric. Generally speaking, any material performing, in a satisfactory manner, the function of receiving the liquid and transferring it into the reservoir layer 11 may be used. Thus, if it is desirable to use a material other than a woven fabric to form the receiving layer, it is necessary to check that the liquid spreads rapidly over the entire surface of the material and not only locally, so that the interface for migration of the liquid into the reservoir layer 11 is maximal. While woven fabrics, due to their weaves, have this property of rapidly spreading any drop of liquid applied thereto, certain non-woven fabrics or porous materials are also likely to have this advantage but should be checked by conducting tests within the understanding of those skilled in the art.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I/we claim:

1. A dermal patch comprising;
a reservoir layer comprising a polymer gel capable of receiving a liquid containing at least one active substance and having a first face to be applied onto skin of a user to diffuse the active substance into the skin and a second face for receiving the liquid,
a receiving layer for receiving the liquid containing the active substance and transmitting the liquid to the reservoir layer, and
a layer of adhesive holding the receiving layer on the second face of the reservoir layer, wherein the adhesive layer does not hinder the transmission of the liquid from the receiving layer to the reservoir layer.

2. The patch according to claim 1, wherein the reservoir layer comprises a polymethylsiloxane resulting from polymerization of a mixture of silicone oils.

3. The patch according to claim 1, wherein the reservoir layer comprises dimethicone or a mixture of dimethicone and vinyl dimethicone.

4. The patch according to claim 1, wherein the receiving layer comprises a fabric which is elastic in at least one direction.

5. The patch according to claim 1, wherein the receiving layer comprises a woven elastane polyamide fabric.

6. The patch according to claim 1, wherein the receiving layer has a basis weight on an order of one hundred to about five hundred grams per square meter and a thickness less than one millimeter.

7. The patch according to claim 1, wherein the reservoir layer has a thickness between 0.2 and 2 mm.

8. The patch according to claim 1, wherein the first face of the reservoir layer is self-adhesive to the skin.

9. The patch according to claim 1, wherein the layer of adhesive has a thickness on an order of one tenth to about five tenths of a millimeter and penetrates as little as possible into the receiving layer.

10. The patch according to claim 1, wherein the layer of adhesive comprises an acetic glue.

11. A method for manufacturing a dermal patch comprising the steps of:
manufacturing a reservoir layer by polymerizing a polymer gel, the reservoir layer being provided for receiving a liquid containing an active substance and diffusing the liquid to the user's skin,
preparing a receiving layer comprising a material capable of receiving the liquid containing the active substance and transmitting the liquid to the reservoir layer, and
assembling the receiving layer and the reservoir layer with an intermediate layer of adhesive, the adhesive layer being formed so as not to hinder the transmission of the liquid from the receiving layer to the reservoir layer.

12. The method according to claim 11, further comprising a step of pouring a dose of the liquid containing an active substance onto the receiving layer.

13. The method according to claim 11, wherein the liquid containing an active substance is mixed with the polymer gel prior to polymerization thereof.

14. The method according to claim 11, wherein the receiving layer comprises a fabric which is elastic in at least one direction.

15. The method according to claim 11, wherein the receiving layer comprises a woven elastane polyamide fabric.

16. The method according to claim 11, wherein the receiving layer has a basis weight of one hundred to about five hundred grams per square meter and a thickness less than one millimeter.

17. The method according to claim 11, wherein the adhesive layer has a thickness on an order of one tenth to about five tenths of a millimeter and penetrates as little as possible into the receiving layer.

18. The method according to claim 11, wherein the reservoir layer comprises a polymethylsiloxane resulting from polymerization of a mixture of silicone oils.

19. The method according to claim 11, wherein the reservoir layer comprises dimethicone or a mixture of dimethicone and vinyl dimethicone.

20. The method according to claim 11, wherein the reservoir layer has a first face to be applied onto the user's skin, the first face being self-adhesive to the skin.

21. The method according to claim 11, wherein the layer of adhesive comprise an acetic glue.
